(19) United States
(12) Patent Application Publication
(10) Pub. No.: US 2022/0344061 A1
(43) Pub. Date: Oct. 27, 2022

(54) BIOLOGICAL KIN RECOGNITION METHOD AND SYSTEM BASED ON UNSUPERVISED CLUSTERING OF mRNA BASE

(71) Applicant: Renmin University of China, Beijing (CN)

(72) Inventors: Xun Liang, Beijing (CN); Zihuan Feng, Beijing (CN); Weilan Huang, Beijing (CN)

(21) Appl. No.: 17/349,851

(22) Filed: Jun. 16, 2021

(30) Foreign Application Priority Data
Apr. 23, 2021 (CN) 202110440432.4

(51) Int. Cl. G16H 50/70; G16H 30/40; G06N 3/04; G06N 3/08

(52) U.S. Cl. CPC G16H 50/70; G16H 30/40; G06N 3/04; G06N 3/088

(57) ABSTRACT

The present disclosure belongs to the technical field of intelligent kin recognition, and relates to a new biological kin recognition method and system based on unsupervised clustering of mRNA bases, including the following steps: step S1, extracting base codons from an mRNA chain, and re-encoding the base codons according to encoding rules; step S2, converting a re-encoded base chain into a document capable of being identified by a model; step S3, inputting the document into the model to vectorize base texts, and clustering vectorized base texts; and step S4, visualizing clustering results to obtain a biological kin recognition result. The present disclosure does not need to artificially annotate the data, saves labor costs, and avoids effects of artificial factors on taxonomical results, featuring simple use, efficient program run, and fast speed.

### CROSS REFERENCE TO RELATED APPLICATION

This patent application claims the benefit and priority of Chinese Patent Application No. 202110440432.4, filed on Apr. 23, 2021, the disclosure of which is incorporated by reference herein in its entirety as part of the present application.

### TECHNICAL FIELD

The present disclosure relates to a new biological kin recognition method and system based on unsupervised clustering of mRNA bases, and belongs to the technical field of intelligent kin recognition, and in particular to the technical field of intelligent biological kin recognition conducted based on bases.

### BACKGROUND ART

Messenger RNA (mRNA), transcribed from a strand as a template, is a single-stranded RNA that carries genetic information to direct the protein synthesis. The mRNA delivers the genetic information from DNA to ribosome, in which the mRNA serves as a template for protein synthesis and determines an amino acid sequence of the peptide chain of the protein product after gene expression. Using a cell gene as a template, once an mRNA is transcribed and produced according to the base complementation pairing rule, the mRNA has base sequences corresponding to some functional fragments in a DNA molecule, and serves as a direct template for protein biosynthesis. For example, like DNA, the genetic information of mRNA is stored in a nucleotide sequence, and is arranged in a codon composed of three bases. Each codon encodes a specific amino acid; however, stop codon is an exception because the stop codon terminates the protein synthesis.

Development of mRNA vaccines indicates that mRNAs carries some information of viruses, the genetic information in mRNAs is preserved in nucleotide sequences, and different nucleotide sequences represent different viruses. In recent years, outbreaks of various epidemic viruses cause substantial inconvenience to life and production, threaten to people's life and health, and cause substantial economic loss. It has been found that a plurality of viruses come from nature or variants of some viruses existing in nature, and even there are strong similarities among a plurality of viruses. Therefore, kin recognition among viruses becomes an urgent problem to be solved in the prior art. So far, kin recognition methods used in the biology community do not take the role of mRNA into account, and have shortcomings of laborious steps, long duration, strong dependence on equipment use, frequent manual intervention, and high labor costs. The method set forth by the present disclosure adopts a computer program to enrich and expand the application mode and research field of mRNAs in the biology community, and will further provide an effective and multifunctional practical tool for future research and source tracing of genes of infectious viruses.

### SUMMARY

In view of the problems, an objective of the present disclosure is to provide a new biological kin recognition method and system based on unsupervised clustering of mRNA bases. The present disclosure does not need to artificially annotate the data, saves labor costs, and avoids effects of artificial factors on taxonomical results, featuring simple use, efficient program run, and fast speed.

To achieve the above objective, the present disclosure adopts the following technical solutions: a biological kin recognition method based on unsupervised clustering of mRNA bases, including the following steps: step S1, extracting base codons from an mRNA chain, and re-encoding the base codons according to encoding rules; step S2, converting a re-encoded base chain into a document capable of being identified by a model; step S3, inputting the document into the model to vectorize base texts, and clustering vectorized base texts; and step S4, visualizing clustering results to obtain a biological kin recognition result.

Further, the encoding in step S1 may be to characterize four bases by means of two-digit secondary codes.

Further, in step S2, the document capable of being identified by a model may be converted by the re-encoded base chain in the form of content mapping, and the document may include names of creatures represented by mRNA chains and the corresponding base chain codes.

Further, in step S3, a method for inputting the document into the model to vectorize base texts may include the following steps: step S3.1, confirming two parameters, optimal sliding window and dimension of model construction, in document embedding; and step S3.2, conducting manifold learning on a normalized vector normalized vector of each document, conducting dimension reduction on the normalized vector, and converting a higher dimensional matrix into a two-dimensional vector group to reduce a high-dimensional image to a two-dimensional one.

Further, in step S3.1, a method for confirming the optimal sliding window and the dimension of model construction may include the steps of: constructing document embedding models in different dimensions to obtain document-embedded matrices, calculating model losses in all dimensions according to the matrices, and minimizing the model losses to obtain an optimal window; plotting noises of a loss function calculation model in the optimal window to obtain broken line graphs of the model in different dimensions and thus an optimal dimension of model construction; and verifying the optimal window via the optimal dimension of model construction.

Further, specific steps for obtaining the optimal window may include: fixing a window or a dimension; calculating a document-embedded matrix A, and traversing the window or the dimension to obtain a set of matrices {A}; for any matrix M₁ in the set of matrices {A}, calculating SUMDVL=SUM(DVL(M₁Nₒₜₕₑᵣ)), where Mₒₜₕₑᵣ is other matrix other than M₁ in a set {X}; and using a window with minimal SUMDVL as the optimal window.

Further, the model may be an unsupervised deep learning model Doc2Vec.

Further, in S3.2, the dimension reduction of normalized vector may include the following steps: looking for a mapping relationship ƒ of a dataset aᵢ in high-dimensional space, constructing a low-dimensional dataset {yᵢ=f(aᵢ)} according to the mapping relationship ƒ, and reducing a high-dimensional vector to a two-dimensional one through a nonlinear T-SNE in the manifold learning, to obtain a cluster visualization result.

Further, the two-dimensional vector in each document may be regarded as a scattered point and plotted to a cluster visualization result graph; in the cluster visualization result graph, if the distance between two scattered points is lower than a threshold, both scattered points may have a genetic relationship, otherwise, neither one may have a genetic relationship.

The present disclosure further provides a biological kin recognition system based on mRNA bases, including: a re-encoding module, used for extracting base codons from an mRNA chain, and re-encoding the base codons according to encoding rules; a conversion module, used for converting a re-encoded base chain into a document capable of being identified by a model; a clustering module, used for inputting the document into the model to vectorize base texts, and clustering vectorized base texts; and a display module, used for visualizing clustering results to obtain a biological kin recognition result.

The present disclosure has the following advantages:

1. A core algorithm of the present disclosure is an unsupervised algorithm, which does not need to artificially annotate the data before use, saves labor costs, avoids effects of artificial factors on taxonomical results, and is used for taxonomical identification of unknown creatures. From the computer's point of view, the present disclosure provides a new basis for biological genetic evolution, featuring simple use, efficient program run, and fast speed.

2. The method in the present disclosure may be widely used, which may be used for not only the taxonomical identification of mRNAs, but also for the discrimination of other data lacking annotation information in real life.

### BRIEF DESCRIPTION OF THE DRAWINGS

FIG. 1 is a flowchart of a biological kin recognition method based on mRNA bases in an embodiment of the present disclosure;

FIG. 2 illustrates a model of a kin recognition method of creatures with single-stranded mRNA in an embodiment of the present disclosure;

FIG. 3 illustrates a visual interface of the kin recognition of creatures with single-stranded mRNA in an embodiment of the present disclosure.

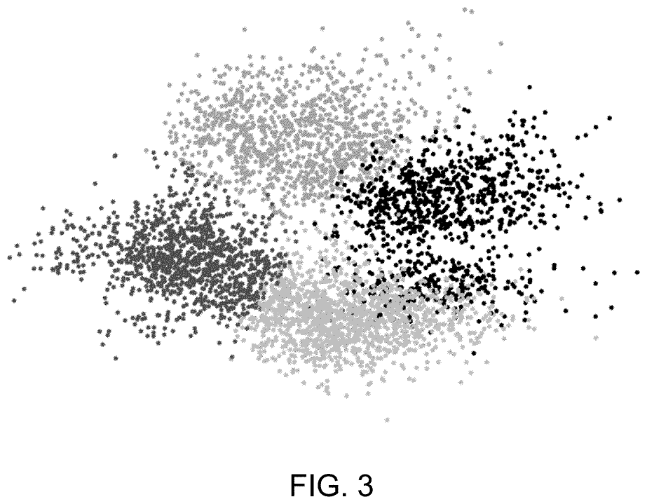

### DETAILED DESCRIPTION OF THE EMBODIMENTS

To enable those skilled in the art to better understand the technical direction of the present disclosure, the present disclosure will be described in detail in conjunction with specific examples. However, it should be understood that provision of specific implementation is only intended to better understand the present disclosure, and they should not be construed as limiting the present disclosure. In the description of the present disclosure, it should be understood that the terms used herein are for descriptive purposes only and cannot be construed as indicating or implying relative importance.

The present disclosure provides a new biological kin recognition method and system based on mRNA bases. In the present disclosure, nucleotide sequences are extracted by biological means, and a plurality of different sequences are analyzed by means of the computer; by re-encoding nucleotide sequences of different mRNAs, documents formed by new encoding are input into a computer model to train a neural network model, extract biological characteristics of RNAs represented by the sequences, collect sequence chains with similarity or genetic relationship together, and realize the identification of biological affinity in the field of computer. The solutions of the present disclosure will be described in detail below in conjunction with two embodiments.

### Embodiment 1

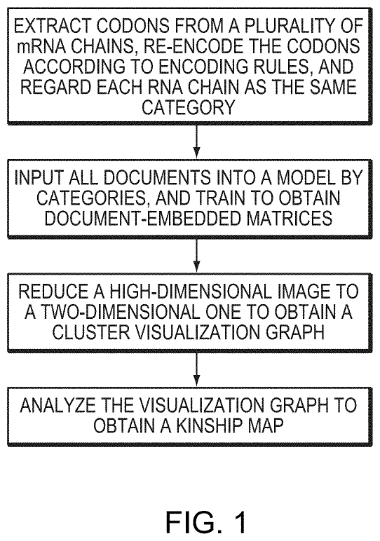

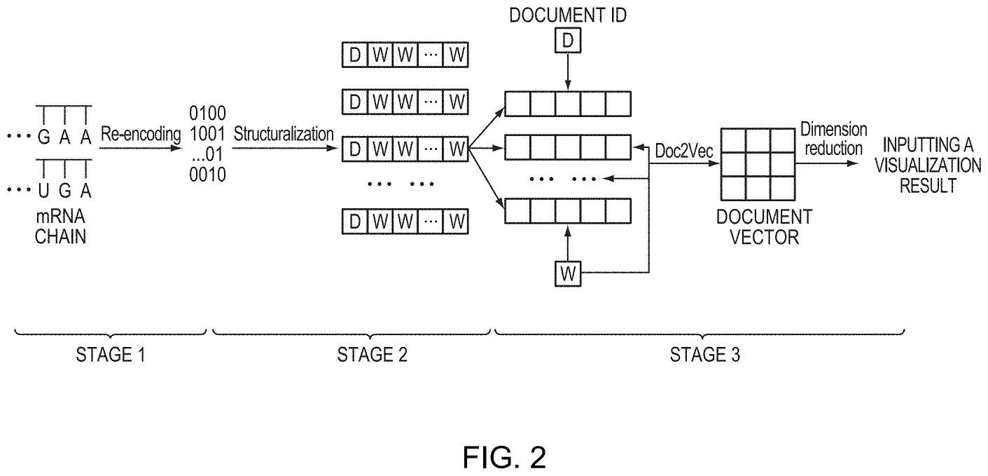

This embodiment disclosed a biological kin recognition method based on unsupervised clustering of mRNA bases, as shown in FIGS. 1 and 2, including the following steps:

Step S1, base codons were extracted from an mRNA chain, and re-encoded according to encoding rules.

Specific steps were as follows: a plurality of mRNA chains were obtained by biological means, and a base codon was extracted from a segment of mRNA to produce a base chain; meanwhile, a well-compiled base transcoding program was used to convert bases into the corresponding computer-recognizable coding forms composed of 0 and 1. In this embodiment, codes were extended from one digit to two digits, and “00”, “01”, “10”, and “11” were used to characterize codes of four bases that constitute RNAs. The four bases had the following encoding schemes: A (adenine) was re-encoded as “00”, G (guanine) as “01”, C (cytosine) as “10”, and U (uracil) as “11”.

Step S2, a re-encoded base chain was converted into a document capable of being identified by a model, i.e., a document in txt format, and text transformation was realized by content mapping. The mRNA information of a creature was composed of a plurality of documents. Herein, text title, i.e., unique identification code of the text, was a name of a creature represented by mRNA chain; text content was a code of the corresponding base chain, and the length of the base chain included in each text was 120. It should be noted that both the txt document and the specific length of the base chain herein were preferred solutions of this embodiment, but use of documents in other formats in a neural network model was not excluded, and the base chain might also have other lengths. Herein, the model was preferably a neural network model, and more preferably an unsupervised deep learning model Doc2Vec, but other applicable models were not excluded.

Step S3, the document was input into the model to vectorize base texts, and vectorized base texts were clustered; mRNA structures represented by bases were identified by introducing priori knowledge and clustering methods.

A method for inputting the document into the model to vectorize base texts included the following steps: Step S3.1, two parameters, optimal sliding window and dimension of model construction, were confirmed in document embedding.

In step S3.1, a method for confirming the optimal sliding window and the dimension of model construction included the following steps: document embedding models in different dimensions were constructed to obtain document-embedded matrices, model losses in all dimensions were calculated according to the matrices, and the model losses were minimized to confirm windows with a fixed dimension value of 230 and thus to obtain an optimal window; subsequently, noises of a loss function calculation model were plotted in the optimal window to obtain broken line graphs of the model in different dimensions, and thus an optimal dimension of model construction was obtained; the optimal window was verified via the optimal dimension of model construction.

Specific steps for obtaining the optimal window included as follows: a window or a dimension was fixed; a document-embedded matrix A was calculated, and the window or the dimension was traversed to obtain a set of matrices {A}; for any matrix M₁ in the set of matrices {A}, SUMDVL=SUM(DVL(M₁, Mₒₜₕₑᵣ)) was calculated, where Mₒₜₕₑᵣ is other matrix other than M₁ in a set {X}, and using a window with minimal SUMDVL as the optimal window.

Step S3.2, manifold learning was conducted on a normalized vector inputting model of each document, dimension reduction was conducted on the normalized vector, and a higher dimensional matrix was converted into a two-dimensional vector group to reduce a high-dimensional image to a two-dimensional one.

In S3.2, the dimension reduction of normalized vector included the following steps: a mapping relationship ƒ of a dataset aᵢ was looked for in high-dimensional space, and a low-dimensional dataset {yᵢ=f(aᵢ)} was constructed according to the mapping relationship ƒ, where {yᵢ} net the given conditions in dimensions. A high-dimensional vector was reduced to a two-dimensional one through a nonlinear T-SNE in the manifold learning, and a cluster visualization result was obtained. Nonlinear dimension reduction method had considered both the distance and the topology of the mapping data; use of the nonlinear dimension reduction method in the document-embedded matrix of the high-dimensional data could preserve original features of the vector data, while the low-dimensional data obtained could be visualized.

The two parameters were confirmed successively by controlling variables; document embedding models in different dimensions were constructed to obtain document-embedded matrices in different dimensions, and model loss in each dimension was calculated according to the matrices. After the optimal window was confirmed, noises of a loss function calculation model were plotted in the fixed window to obtain broken line graphs of the model in different dimensions, and an optimal dimension was confirmed. After the optimal dimension was confirmed, the window was verified again.

After the optimal dimension and the optimal window were confirmed, all documents were input into the model by categories and trained, and normalized transformation was conducted on vectors to obtain document-embedded matrices.

Step S4, clustering results were visualized to obtain a biological kin recognition result.

As shown in FIG. 5, the two-dimensional vector in each document is regarded as a scattered point and plotted to obtain a cluster visualization result graph; in the cluster visualization result graph, if the distance between two scattered points is lower than a threshold, both scattered points may have a genetic relationship, otherwise, neither one may have a genetic relationship. If two types of points mess up, this will represent that there is a certain similarity between mRNA bases represented by the two types of points, and will further demonstrate that there is a certain relationship, and even a genetic relationship, between RNAs represented by mRNAs; if two cliques gathered by scattered points are far away on the coordinate system, it may be indicated that there is no relationship between creatures with these two RNA types.

### Embodiment 2

Based on the similar inventive concept, this embodiment disclosed a biological kin recognition system based on unsupervised clustering of mRNA bases, including:

a re-encoding module, used for extracting base codons from an mRNA chain, and re-encoding the base codons according to encoding rules;

a conversion module, used for converting a re-encoded base chain into a document capable of being identified by a model;

a clustering module, used for inputting documents into the model to vectorize base texts, and clustering vectorized base texts; and

a display module, used for visualizing clustering results to obtain a biological kin recognition result.

Finally, it should be noted that: the above embodiments are merely intended to describe the technical solutions of the present disclosure, rather than to limit thereto; although the present disclosure is described in detail with reference to the above embodiments, it is to be appreciated by a person of ordinary skill in the art that modifications or equivalent substitutions may still be made to the specific implementations of the present disclosure, and any modifications or equivalent substitutions made without departing from the spirit and scope of the present disclosure shall fall within the protection scope of the claims of the present disclosure. The above merely describes specific implementations of the present application, but the protection scope of the present application is not limited thereto. Any modifications or replacements easily conceived by those skilled in the art within the disclosed technical scope of the present application shall fall within the protection scope of the present application. Therefore, the protection scope of the present application shall be subject to the protection scope of the claims.

1. A biological kin recognition method based on unsupervised clustering of mRNA bases, comprising the following steps:
step S1, extracting base codons from an mRNA chain, and re-encoding the base codons according to encoding rules;
step S2, converting a re-encoded base chain into a document capable of being identified by a model;
step S3, inputting the document into the model to vectorize base texts, and clustering vectorized base texts; and
step S4, visualizing clustering results to obtain a biological kin recognition result.

2. The biological kin recognition method based on mRNA bases according to claim 1, wherein the encoding in step S1 is to characterize four bases by means of two-digit secondary codes.

3. The biological kin recognition method based on mRNA bases according to claim 1, wherein in step S2, the document capable of being identified by a model are converted by the re-encoded base chain in the form of content mapping, and the document comprises names of creatures represented by mRNA chains and the corresponding base chain codes.

4. The biological kin recognition method based on mRNA bases according to claim 1, wherein in step S3, a method for inputting the document into the model to vectorize base texts comprises the following steps:
step S3.1, confirming two parameters, optimal sliding window and dimension of model construction, in document embedding; and
step S3.2, conducting manifold learning on a normalized vector normalized vector of each document, conducting dimension reduction on the normalized vector, and converting a higher dimensional matrix into a two-dimensional vector group to reduce a high-dimensional image to a two-dimensional one.

5. The biological kin recognition method based on mRNA bases according to claim 4, wherein in step S3.1, a method for confirming the optimal sliding window and the dimension of model construction comprises the steps of: constructing document embedding models in different dimensions to obtain document-embedded matrices, calculating model losses in all dimensions according to the matrices, and minimizing the model losses to obtain an optimal window; plotting noises of a loss function calculation model in the optimal window to obtain broken line graphs of the model in different dimensions and thus an optimal dimension of model construction; and verifying the optimal window via the optimal dimension of model construction.

6. The biological kin recognition method based on mRNA bases according to claim 5, wherein specific steps for obtaining the optimal window comprise: fixing a window or a dimension; calculating a document-embedded matrix A, and traversing the window or the dimension to obtain a set of matrices {A}; for any matrix M₁ in the set of matrices {A}, calculating SUMDVL=SUM(DVL(M₁, Mₒₜₕₑᵣ)), wherein Mₒₜₕₑᵣ is other matrix other than M₁ in a set {X}; and using a window with minimal SUMDVL as the optimal window.

7. The biological kin recognition method based on mRNA bases according to claim 6, wherein the model is an unsupervised deep learning model Doc2Vec.

8. The biological kin recognition method based on mRNA bases according to claim 4, wherein in step S3.2, the dimension reduction of the normalized vector comprises the following steps: looking for a mapping relationship ƒ of a dataset aᵢ in high-dimensional space, constructing a low-dimensional dataset {yᵢ=f(aᵢ)} according to the mapping relationship ƒ, and reducing a high-dimensional vector to a two-dimensional one through a nonlinear T-SNE in the manifold learning, to obtain a cluster visualization result.

9. The biological kin recognition method based on mRNA bases according to claim 8, wherein the two-dimensional vector in each of the document is regarded as a scattered point and plotted to a cluster visualization result graph; in the cluster visualization result graph, if the distance between two scattered points is lower than a threshold, both scattered points have a genetic relationship, otherwise, neither one has a genetic relationship.

10. A biological kin recognition system based on unsupervised clustering of mRNA bases, comprising:
a re-encoding module, used for extracting base codons from an mRNA chain, and re-encoding the base codons according to encoding rules;
a conversion module, used for converting a re-encoded base chain into a document capable of being identified by a model;
a clustering module, used for inputting the document into the model to vectorize base texts, and clustering vectorized base texts; and
a display module, used for visualizing clustering results to obtain a biological kin recognition result.
